(19) United States
(12) Patent Application Publication
(10) Pub. No.: US 2025/0143996 A1
(43) Pub. Date: May 8, 2025

(54) TWO-IN-ONE BODY SCRUB AND SHAVE PREPARATION

(71) Applicant: Edgewell Personal Care Brands, LLC, Chesterfield, MO (US)

(72) Inventors: Stacie Cobb, East Haven, CT (US); Rooma Mehta, Guilford, CT (US)

(21) Appl. No.: 18/839,793

(22) PCT Filed: Mar. 2, 2023
(86) PCT No.: PCT/US2023/063568

§ 371 Date: Aug. 20, 2024

Related U.S. Application Data
(60) Provisional application No. 63/315,775, filed on Mar. 2, 2022.

(51) Int. Cl. A61K 8/60; A61K 8/02; A61K 8/25; A61K 8/92; A61Q 9/02; A61Q 19/00

(52) U.S. Cl. CPC A61K 8/60; A61K 8/0241; A61K 8/25; A61K 8/922; A61Q 9/02; A61Q 19/00; A61K 2800/28; A61K 2800/31; A61K 2800/412; A61K 2800/413

(57) ABSTRACT

Embodiments relate to a topical composition including an exfoliation particle selected to break down or dissolve when the topical composition is rubbed on skin of a body part. The topical composition further includes a cosmetically acceptable carrier to disperse the exfoliating particle in the topical composition and deliver the exfoliating particle to the skin or the body part. The topical composition is formulated to exfoliate skin during an exfoliation process and subsequently enhance shave quality during a shave process involving the body part by transformation from a body scrub into a shave preparation during use as a two-in-one body scrub and shave preparation composition.

### CROSS-REFERENCE TO RELATED APPLICATIONS

This application claims priority to U.S. Provisional Patent Application No. 63/315,775, filed on Mar. 2, 2022, the content of which is incorporated herein for reference in its entirety.

### TECHNICAL FIELD

Embodiments relate to a topical composition including an exfoliation particle and a cosmetically acceptable carrier. The topical composition is formulated to provide a body scrub and a shave preparation in a single composition (two-in-one composition).

### BACKGROUND

Technologies are available to exfoliate skin. For example, commercially available scrub products are traditionally formulated to thin out during application so as to easily be wash away once the exfoliation process is complete. Thus, no consideration in design is given to performance of the composition after the exfoliation process. Such scrub products are also traditionally formulated to provide a relatively large quantity of exfoliation particles and/or to increase the residence time of exfoliation particles during use to maximize exfoliation. Thus, the sensory profile that is provided remains unfavorable to consumers for a shave process and drives consumers to wash such scrub products away after the exfoliation process.

Meanwhile, commercially available shave preparations that are applied to skin before a shave process are traditionally formulated to avoid ingredients that can impart unfavorable sensory attributes during the shave process or that can interfere with shave comfort (e.g., glide, etc.). Even when shave preparations include microbeads, such shave preparations are not formulated for a breakdown of the microbeads and therefore provide a sensory profile that might remain unfavorable to consumers throughout a shave process. Commercially available scrub products and shave preparations, therefore, are traditionally independent products addressing different problems or consumer needs with ingredients that are considered to be technically or commercially impractical to combine. Thus, there is considerable room for improvement to provide a topical composition that is formulated to provide a body scrub and a shave preparation in a single composition.

### SUMMARY

Embodiments relate to a topical composition including an exfoliation particle to exfoliate skin on a body part. The exfoliation particle is selected to break down or dissolve when the topical composition is used on the body part. The topical composition further includes a cosmetically acceptable carrier to disperse the exfoliating particle and deliver the exfoliating particle to the body part. Notably, the topical composition is formulated to transform from a body scrub into a shave preparation. Accordingly, the technical solutions encompassed by aspects of embodiments may be useful to minimize the unnecessary inconvenience to consumers or waste of resources from using multiple products by providing a topical composition that is effective to exfoliate the skin during an exfoliation process and that is subsequently effective to enhance shave comfort using the same topical composition.

Embodiments relate to a topical composition including an exfoliation particle comprising a water-insoluble particle, a water-soluble particle, or combinations thereof. The topical composition further includes a cosmetically acceptable carrier. In one example, the topical composition is in the form of an emulsion or an anhydrous base. An emulsion may include an aqueous phase and a non-aqueous phase, wherein the water-insoluble particle may be dispersed in the emulsion. In another example, the water-insoluble particle and/or the water-soluble particle may be dispersed in the anhydrous base. Preferably, the emulsion and/or the anhydrous base further include a cleansing surfactant. In one example, the cleansing surfactant may be present in an amount of about 10.0 wt. % to about 50.0 wt. %, based on the total weight of the topical composition.

Embodiments relate to a method to exfoliate the skin of the body part during an exfoliation process and to provide shave comfort to the skin of the body part during a shave process. The method may include contacting the skin with the topical composition, rubbing the topical composition on the skin to exfoliate the skin during the exfoliation process, and applying a shave device to the skin during the shave process subsequent to the exfoliation process. Optionally, the method may include contacting the topical composition with water. In one example, a user may rub the topical composition on the skin for a period of time within which mechanical force breaks down a majority of water-insoluble particles and/or the water dissolves a majority of water-soluble particles. The period of time may be sufficient to allow the cleansing surfactant to mix with the water to provide a lather effect that causes the topical composition to remain on the skin and/or to provide better shave comfort during the shave process (compared to a topical composition having the exfoliation particles with less surfactant or no cleansing surfactant that is used for the same amount of time). In another example, the period of time may be sufficient to allow the topical composition to remain present on the skin to provide at least some shave comfort during the shave process.

Embodiments relate to making the topical composition, which may include mixing the exfoliation particle, the cosmetically acceptable carrier, and optionally one or more other ingredients. Traditional methods may be implemented to provide various forms including, without limitation, emulsions, lotions, creams, anhydrous bases, or combinations thereof. Embodiments may include various consumer packaged products such as, without limitation, topical compositions in containers, tubes, jars, etc., accessed via caps, pumps, sprays, etc.

### BRIEF DESCRIPTION OF THE DRAWINGS

The features of the disclosure believed to be novel and the elements characteristic of the invention are set forth with particularity in the appended claims. The figures are for illustration purposes only and are not drawn to scale. The disclosure itself, however, both as to organization and method of operation, can best be understood by reference to the description of the preferred embodiment(s) which follows, taken in conjunction with the accompanying drawings in which:

FIG. 1A to FIG. 1B shows an example of a topical composition that is formulated to transform from a body scrub to a shave preparation according to embodiments.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %.) based on a total weight of the composition unless otherwise indicated. Where appropriate, the International Nomenclature of Cosmetic Ingredients (INCI) name of ingredients/components is provided. In this regard, the term “(and)” is an INCI designation for a mixture of listed components separated by the term. Any numerical range recited herein is intended to include all sub-ranges subsumed therein, and such ranges are understood to include each and every number and/or fraction between the stated range lower and upper values. Moreover, the term “about” refers to deviations of +10%.

### Exfoliation Particle

Topical compositions include exfoliation particles to exfoliate the skin of a body part such as a foot, a leg, a back, a torso, an arm, a face, a part thereof, or combinations thereof. For example, an exfoliation particle may be used to exfoliate an upper lip, a cheek, a neck, a chin, a shin, and so on, or combinations thereof. Generally, an exfoliation process involves applying a topical composition including one or more types of exfoliation particles on the surface of skin and rubbing the topical composition to allow the exfoliation particles to exfoliation the skin. Notably, exfoliation can improve the look and feel of the skin. Exfoliation can also help to release hairs trapped just below the follicle or orient hairs on the skin just before the hairs are cut by a razor blade, leading to a closer and more efficient shave.

Exfoliation particles may, therefore, include any material suitable to exfoliate the skin. Useful exfoliation particles may include, without limitation, fruit seeds, fruit stones, nut shells, ground or fibrous plant material, polymers, mineral composites, or combinations thereof. For example, exfoliation particles may include, without limitation, ground coconut shells, ground apricot seeds, ground peach seeds, ground olive seeds, ground walnut shells, ground almond shells, ground pecan shells, ground luffa, corn cob granules, ground oatmeal, polymer beads or granular polymers, jojoba wax beads, rice bran, silica (hydrated, compacted, etc.), sand, minerals, pumice sand, clay, carbohydrates, or combinations thereof.

Exfoliation particles in embodiments may be water-insoluble, such as silica, or may be water-soluble, such a saccharide (monosaccharides, disaccharides, etc.). For example, exfoliation particles that are water-soluble may include natural sugars such as trehalose, sucrose, glucose, and so on, or combinations thereof. However, artificial sugars (e.g., sucralose, etc.) may also be useful in embodiments. Generally, water-insoluble exfoliation particles may be broken down my mechanical force (e.g., shear, etc.) when a consumer rubs a topical composition (such as a body scrub product) on the skin. Conversely, water-soluble exfoliation particles dissolve when mixed with water. In one embodiment, sucrose may be selected from the group consisting or white sugar, brown sugar (e.g., coconut sugar, etc.), or combinations thereof. For example, the weight ratio of white sugar to brown sugar may be about 1:50 to about 50:1, such 1:1, 1:5, 1:10, 1:15, 1:20, 1:30, 1:40, 1:50, 1:55, 55:1, 40:1, 30:1, 25:1, 20:1, 15:1, 10:1, 5:1, including every number and/or fraction therebetween. The same weight ratios may be used for other combinations of different exfoliation particles.

Additionally or alternatively, exfoliation particles may have an average particle size from about 10 nm to about 1 μm. For example, an exfoliation particle may have an average particle size of 8 nm, 25 nm, 50 nm, 75 nm, 100 nm, 200 nm, 300 nm, 400 nm, 500 nm, 600 nm, 700 nm, 800 nm, 900 nm, 1.2 μm, including every number and/or fraction therebetween. For example, silica particles may have an average particle size from about 10 nm to about 500 nm while sugar particles may have an average particle size from about 450 nm to about 1 μm. Exfoliation particles may also have a variety of shapes such as spherical, elliptical, amorphous, plate-like, etc. In one example, exfoliation particles are granular solid spherical particles.

Exfoliation particles may be present from about 1.0 wt. % to about 40.0 wt. %, based on the total weight of the topical composition. An exfoliation particle may, for example, be present in an amount of 0.8 wt. %, 1.0. %, 2.0 wt. %, 3.0 wt. %, 3.5 wt. %, 5.0 wt. %, 10.0 wt. %, 15.0 wt. %, 17.0 wt. %, 20.0 wt. %, 25.0 wt. %, 30.0 wt. %, 35.0 wt. %, 44.0 wt. %, including every number and/or fraction therebetween, based on the total weight of the topical composition. For example, silica may be present in an amount of about 1.0 wt. % to about 10.0 wt. %, based on the total weight of the topical composition. Additionally or alternatively, sucrose may be present in an amount of about 2.0 wt. % to about 35.0 wt. %, such as from 20 wt. % to 30 wt. % for example, based on the total weight of the topical composition.

### Cosmetically Acceptable Carrier

Topical compositions may be prepared in various forms such as, without limitation, oils, emulsions, lotions, creams, anhydrous bases, or combinations thereof. Ingredients and use levels to make such formats may be readily adjusted and mixed in a single phase or in multiple phases to provide a desired commercial personal care product. Thus, ingredients in topical compositions may be readily included, omitted, adjusted, or mixed in a single phase or in multiple phases to provide a commercial personal care product that accounts for cost, aesthetics, and/or product performance objectives. Moreover, topical compositions can be filled into any number of bottles, tottles, tubes, etc., having a variety of closing structures.

Accordingly, topical compositions may be composed of a cosmetically acceptable carrier including, without limitation, water, vegetable oil, plant oil, esters (e.g., octyl palmitate, isopropyl myristate, isopropyl palmitate, etc.), ether (e.g., dicapryl ether, dimethyl isosorbide, etc.), alcohols (e.g., ethanol, isopropanol, etc.), fatty alcohol (e.g., behenyl alcohol, etc.), isoparaffin (e.g., isooctane, isododecane, isohexadecane, etc.), silicone oil (e.g., dimethicones, polysiloxanes, etc.), hydrocarbon oil (e.g., mineral oil, petrolatum, isoeicosane polyisobutene, etc.), polyol (e.g., propylene glycol, glycerin, butylene glycol, pentylene glycol, hexylene glycol, etc.), wax (e.g., vegetable, animal, silicone), butter, or combinations thereof.

For example, topical compositions may be composed of a skin-conditioning agent (e.g., emollients, humectants, etc.). Examples of useful skin-conditioning agents include, without limitation, esters (e.g., isopropyl myristate, isopropyl palmitate, 1-decene polymer (hydrogenated), esters of benzoic acid such as lauryl/myristyl benzoate, C₁₂-C₁₅ alkyl benzoate, C₁₆-C₁₇ alkyl benzoate, stearyl benzoate, isostearyl benzoate, behenyl benzoate, ethylhexyl benzoate, butyloctyl benzoate, hexyldecyl benzoate, octyldodecyl benzoate, etc.), polyols (e.g., glycerin, butylene glycol, hexylene glycol, 2,3-butanediol, 1,2-alkane diols such propylene glycol, 1,2-butanediol, pentylene glycol, ethyl hexanediol, caprylyl glycol, decylene glycol, etc.), natural oils (e.g., nut oil, seed oil, fruit oil, etc.), fatty acids (e.g., lanolin, petrolatum, mineral oil, etc.), triglycerides (caprylic/capric triglyceride, etc.), silicones (cyclomethicone, dimethicone, etc.), salicylates (e.g., butyloctyl salicylate, C₁₂-C₁₅ alkyl salicylate, capryloyl salicylic acid, hexydodecyl salicylate, isocetyl salicylate, isodecyl salicylate, tridecyl salicylate, etc.), butters (e.g., mango seed, shea, etc.), or combinations.

Preferably, a skin-conditioning agent includes at least one oil. In one example, the oil is selected form Vitis vinifera (grape) seed oil, Cocos nucifera (coconut) oil, mineral oil, or combinations thereof. Additionally or alternatively, any other oils may be useful such as, without limitation, hydrogenated vegetable oil, Olea europaea (oilive) oil, or combinations thereof. Thus, for example, a topical composition may include Vitis vinifera (grape) seed oil present in an amount of about 15.0 wt. % to about 50.0 wt. %, such as about 20.0 wt. % to about 40.0 wt. %, based on the total weight of the topical composition. However, Vitis vinifera (grape) seed oil may be present in an amount less than 15.0 wt. %. For example, a topical composition may include Vitis vinifera (grape) seed oil present in an amount of about 3.0 wt. % or less, based on the total weight of the topical composition. Similarly, Vitis vinifera (grape) seed oil may be present in an amount greater than 50.0 wt. %. For example, a topical composition may include Vitis vinifera (grape) seed oil present in an amount of about 50.0 wt. % to about 80.0 wt. %, based on the total weight of the topical composition. Moreover, Vitis vinifera (grape) seed oil may be replaced, in part or in whole, by one or more other oils such as mineral oil. Thus, in one example, mineral oil may be present in an amount of about 5.0 wt. % to about 20.0 wt. %, based on the total weight of the topical composition.

Water may also be intentionally added in a topical composition at an amount greater than 15.0 wt. % such as, for example, 25.0 wt. %, 45.0 wt. %, 50.0 wt. %, 55.0 wt. %, 60.0 wt. %, 65.0 wt. %, including every number and/or fraction therebetween, based on the total weight of a hydrous topical composition. For example, a water-in-oil (W/O) emulsion may have an aqueous dispersed phase stabilized by an emulsifier and disposed in a hydrophobic continuous phase. Water may, however, be intentionally added in an amount of 15.0 wt. % or less, based on a total weight of the topical composition, to form an anhydrous topical composition. Moreover, one or more ingredients may have associated with it absorbed and/or adsorbed water that can be carried into the otherwise substantially anhydrous personal care composition. For economic, sourcing, and/or formulation reasons, it may be disadvantageous to process hygroscopic materials to make them completely free of water. Thus, an anhydrous base including a non-aqueous carrier and a surfactant may include 15.0 wt. % or less, preferably 5.0 wt. % or less (e.g., ≤1.0 wt. %), of water based on a total weight of the topical composition. An anhydrous topical composition may also be water-free.

Generally, carriers may be present from about 0.1 wt. % to about 95.0 wt. %, based on the total weight of the topical composition. A carrier may, for example, be present in an amount of 0.008 wt. %, 0.1 wt. %, 0.5 wt. %, 1.0 wt. %, 2.0 wt. %, 6.0 wt. %, 10.0 wt. %, 15.0 wt. %, 18.0 wt. %, 20.0 wt. %, 35.0 wt. %, 37.5, 50.0 wt. %, 74.0 wt. %, 82.0 wt. %, 90.0 wt. %, including every number and/or fraction therebetween, based on the total weight of the topical composition. For example, a total amount of oil in an anhydrous base may present from about 30.0 wt. % to about 80.0 wt. %, such as about 30.0 wt. % to about 60.0 wt. %, based on the total weight of the topical composition. In another example, a total amount of water in an emulsion may present from about 30.0 wt. % to about 80.0 wt. %, such as about 50.0 wt. % to about 70.0 wt. %, based on the total weight of the topical composition.

### Surfactant

Surfactants may include anionic, cationic, nonionic, amphoteric, or combinations thereof. Examples of useful anionic surfactants in embodiments include, without limitation, carboxylates, amino acid derivatives, alkyl sulphates, alkyl ether sulfates, sulphonates, isethionates, taurates, sulfosuccinates, alkyl sulfoacetates, phosphates, alkyl phosphates, or combinations thereof. For example, anionic surfactants may include salts of fatty acid esters (e.g., having from 12 to 20 carbon atoms, such as sodium methyl cocoyl taurate), salts of fatty acid isethionates (e.g., having from 12 to 20 carbon atoms such as sodium cocoyl isethionate), salts of fatty acid sulfoacetates (e.g., having from 12 to 20 carbons such as sodium lauryl sulfoacetate), disalts of fatty acid sulfosuccinates (e.g., having from 12 to 20 carbons such as disodium laureth sulfosuccinate), or combinations thereof. Thus, for example, a topical composition may include anionic surfactants selected from salts of acylisethionate (e.g., sodium cocoyl isethionate), alkyl sulfonates (e.g., sodium C₁₂-C₁₄ olefin sulfonate, sodium cocomonoglyceride sulfonate, sodium C₁₂-C₁₅ pareth-15 sulfonate), alkyl sulfoacetates (e.g., sodium lauryl sulfoacetate), or combinations thereof.

Examples of useful cationic surfactants include, without limitation, alkylamines, alkylimidazolines, quaternary ammonium compounds (QAC), or combinations thereof. For example, cationic surfactants may include benzethonium, benzalkonium, cetrimide, cetylpyridinium chloride, stearalkonium chloride, dicetyldimonium chloride, alkyl glucoside, cocoamide monoethanolamine, cocoamide, or combinations thereof. Notably, a salt of benzethonium or benzalkonium may also act as a biocide to kill a microorganism such as, for example, a virus, bacteria, or yeast.

Examples of useful nonionic surfactants include, without limitation, alkanolamides, amine oxides, esterified carboxylic acids, ethoxylated alcohols, poloxamers, or combinations thereof. Nonionic surfactants may include alkyl glucoside (e.g., decyl glucoside etc.), cocoamide monoethanolamine, cocoamide diethanolamine, a glycerol alkyl ester, polyethylene glycol, poloxamer (e.g., 337, poloxamer 407, etc.), or combinations thereof. In one example, polyethylene oxide (PEO) that is commercially available as POLYOX™ from The Dow Chemical Company in a variety of molecular weights and particle sizes can be used. As known in the industry and references in POLYOX™ commercial literature, the molecular weight of POLYOX™ polyethylene oxides, such as those, are determined largely by viscosity data. Thus, one or more PEOs may be selected based on a molecular weight (MW) of about 100,000 Daltons (Da) to about 10,000,000 Da, for example to about 5,000,000 Da.

Examples of useful amphoteric/zwitterionic surfactants include, without limitation, betaines, sultaines, cocamphoacetates, or combinations thereof. For example, an amphoteric surfactant may be selected from fatty aminopropionates (e.g., characterized with the general formula R—NH—(CH₂)₂—COONa), fatty iminodipropionates (e.g., characterized with the general formula R—N—((CH₂)₂—COONa)₂), alkyl betaines (e.g., alkyl dimethyl betaines characterized with the general formula R—N—(CH₃)₂—CH₂—COO), alkyl amidopropyldimethylbetaines (e.g., characterized with the general formula R—CO—NH—(CH₂)₃—N—(CH₃)₂—CH₂—COO), imidazoline derived amphoterics, alkyl poly amino carboxylates (e.g., characterized by four or more nitrogen atoms and four or more carboxylic acid groups), or combinations thereof. Thus, a personal care composition may include amphoteric surfactants selected from cocamidopropyl betaine, lauramidopropyl betaine, cocoamido hydroxyl sultaine, sodium cocamphoacetate, sodium methyl cocoyl taurate, or combinations thereof.

Examples of emulsifiers, which are surfactants that stabilize emulsions, may include oil-in-water (O/W) emulsifiers, W/O emulsifiers, or combinations thereof. One way to distinguish emulsifying surfactants from cleansing surfactants may be by respective hydrophilic-lipophilic balance (HLB) values. For example, cleansing surfactants typically have HLB values ≥13, preferably 15 to 20. Additionally or alternatively, emulsifying surfactants may be distinguished from cleansing surfactants by an ability to provide a lather effect when a topical composition having the surfactant is mixed with water. Generally, emulsifying surfactants provide no or substantially no visible lather effect while cleansing surfactants do provide a visible lather effect. When mixed with water, a later effect in a topical composition may include a visually observable foam, a visually observable lather, increased viscosity of the composition, relatively slower thin rate of the composition, or combinations thereof. In embodiments, a cleansing surfactant may increase the time that a topical composition remains on the skin (residence time).

An O/W emulsifier may have an HLB≥7, preferably 8 to 12. Examples of useful O/W emulsifiers include, without limitation, certain sucrose esters (e.g., sucrose stearate, sucrose laurate, etc.), sorbitan esters (e.g., sorbitan laurate, etc.), polyglyceryl esters (e.g., polyglyceryl-3 methylglucose distearate, etc.), ethoxylated fatty ethers and ethoxylated fatty esters having a HLB≥7, or combinations thereof. Examples of useful natural O/W emulsifiers include, without limitation, cetearyl alcohol (and) cetearyl glucoside, glyceryl stearate (and) cetearyl alcohol (and) sodium stearoyl lactylate, cetearyl wheat straw glycosides (and) cetearyl alcohol, cetearyl olivate (and) sorbitan olivate, or combinations thereof. Thus, a personal care composition may include O/W emulsifiers such as glyceryl stearate (and) PEG-100 stearate, cetearyl alcohol (and) coco-glucoside, cetearyl alcohol (and) dicetyl phosphate (and) ceteth-10 phosphate, or combinations thereof.

Meanwhile, a W/O emulsifier may have an HLB of <7, preferably from 3 to 6. Examples of W/O emulsifiers include, without limitation, certain sucrose esters (e.g., sucrose distearate, etc.), sorbitan esters (e.g., sorbitan oleate, sorbitan dioleate, sorbitan stearate, etc.), polyglyceryl esters (e.g., polyglyceryl-4 isostearate, polyglyceryl dimerate isostearate, etc.), silicones (e.g., a copolymer of a silicone, an alkoxylated derivative of a silicone, or combinations thereof, such as cetyl polyethylene glycol (PEG)/polypropylene glycol (PPG)-10/1 dimethicone, lauryl PEG-8 dimethicone, etc.), fatty acid esters (e.g., glyceryl stearate, etc.), or combinations thereof. Examples of useful natural W/O emulsifiers include cetearyl olivate. Thus, a personal care composition may include W/O emulsifiers such as lauryl PEG-8 dimethicone, cetyl PEG/PPG-10/1 dimethicone, or combinations thereof. It is, however, understood that other emulsion formats may similarly be used such as, without limitation, oil-in-oil (O/O) or oil-in-silicon (O/S) emulsions (e.g., castor oil in silicone oil, etc.), O/W/O, etc.

A surfactant may be present from about 0.1 wt. % to about 50.0 wt. %, based on the total weight of the topical composition. A surfactant may, for example, be present in an amount of 0.08 wt. %, 0.2 wt. %, 0.3 wt. %, 0.4 wt. %, 0.5 wt. %, 1.0 wt. %, 1.25 wt. %, 1.5 wt. %, 2.0 wt. %, 3.0. wt. %, 5.0 wt. %, 10.0 wt. %, 12.5 wt. %, 15.0 wt. %, 16.5 wt. %, 20.0 wt. %, 25.0 wt. %, 30.0 wt. %, 32.0 wt. %, 35.0 wt. %, 40.0 wt. %, 45.0 wt. %, 55.0 wt. %, including every number and/or fraction therebetween, based on the total weight of the personal care composition. For example, a topical composition may include a total amount of emulsifying surfactant present in an amount of about 1.0 wt. % to about 5.0 wt. %, based on the total weight of the topical composition. Additionally or alternatively, a topical composition may include a total amount of cleansing surfactant present in an amount of about 10.0 wt. % to about 45.0 wt. %, based on the total weight of the topical composition.

Accordingly, for example, a topical composition may include sodium lauryl sulfoacetate present in an amount about 10.0 wt. % to about 20.0 wt. %, based on the total weight of the topical composition. In another example, a topical composition may include sodium cocyl isethionate present in an amount of about 20.0 wt. % to about 40.0 wt. %, based on the total weight of the topical composition. However, for example in an emulsion form, a topical composition may include an emulsifying surfactant and exclude a cleaning surfactant. Alternatively, the topical composition may include a cleansing surfactant in an amount of about 1.0 wt. % to about 3.0 wt. %, based on the total weight of the topical composition.

### Other Ingredient

Topical compositions may include one or more other ingredients such as, without limitation, film formers, binders/thickeners, antioxidants, extracts, anti-aging actives, amino acids, colorants, pigments, dyes, opacifiers, pH adjusters, fragrances, fillers, preservatives, preservative boosters, chelating agents, stabilizers, sphingolipids, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, antimicrobial agents, skin cooling compounds, skin pollution protectants, skin penetration enhancers, staining agents, depigmenting agents, hypopigmenting agents, extracts (fruit, flower, plant, vegetable), vitamins (e.g., A, C, E, derivatives thereof), alpha and beta hydroxy acids, retinol and its derivatives, or combinations thereof.

Other ingredients may be present in an amount of about 0.0001 wt. % to about 15.0 wt. %, based on the total weight of the topical composition. One or more other ingredients may, for example, be present in an amount of 0.00008 wt. %, 0.01 wt. %, 0.1 wt. %, 0.25 wt. %, 0.5 wt. %, 1.0 wt. %, 2.5 wt. %, 5.0 wt. %, 10.0 wt. %, 15.0 wt. %, 18.0 wt. %, including every number and/or fraction therebetween, based on the total weight of the topical composition.

Accordingly, for example, a film former may be added to provide a substantially homogenous and/or structured hydro-lipidic film on skin owing to its viscosity-building properties. Generally, a film former may be synthesized from two or more different monomers to form copolymers and/or from individual polymer chains that are connected by bridging molecules or crosslinking agents to form crosslinked copolymers known as crosspolymers. A film former may be selected from monomers, copolymers, cross polymers, and/or terpolymers of an organo-silicone hybrid, biopolymer, (meth)acrylate, abietic acid derivative, olefin, silicone, resin, vinyl acetate (VA), vinyl pyrrolidone (VP), maleate, alkyl ester, long chain or short chain carboxylic acid, or combinations thereof. A film former may include, without limitation, crotonic acid/vinyl C₈-C₁₂ isoalkyl esters/VA/bis-vinyldimethicone crosspolymer, acrylate/dimethicone copolymer, VA/butyl maleate/isobornyl acrylate copolymer, methyl dihydroabietate, trimethylsiloxysilicate, acrylate/C₁₂-C₂₂ (meth)acrylate copolymer, C₂₄-C₂₈ alkyldimethylsiloxy trimethylsiloxysilicate, polymethylsilsesquioxane/trimethylsiloxysilicate, polymethylsilsesquioxane, perfluorononyl dimethicone, trifluoropropyldimethylsiloxy/trimethylsiloxy silsesquioxane, PPG-17/isophorone diisocyanate/dimethylol propionic acid copolymer, polyurethane (e.g., 32, 34, 35, 48, etc.), hydrolyzed wheat protein/PVP crosspolymers, maltodextrin/VP copolymer, butylated PVP copolymer, VP/polycarbamylpolyglycol ester, VP/dimethylaminoethylmethacrylate/polycarbamyl polyglycol ester, VP/Dimethiconyl-acrylate/polycarbamyl polyglycol ester, VP/eicosene copolymer, adipic acid/diglycol crosspolymer, trimethylpentanediol/adipic acid/glycerin crosspolymer, polyester-7 (and) neopentyl glycol diheptanoate, polyester-10 (and) propylene glycol dibenzoate, hydrogenated polycyclopentadiene (and) isododecane, shellac, stearyl/octyldodecyl citrate crosspolymer or combinations thereof.

Film formers may be present in an amount of about 0.1 wt. % to about 5.0 wt. %, based on the total weight of the skin care composition. A film former may, for example, be present in an amount of 0.09 wt. %, 0.9 wt. %, 1.0 wt. %, 2.0 wt. %, 3.0 wt. %, 4.0 wt. %, 5.0 wt. %, 5.5 wt. %, including every number and/or fraction therebetween, based on the total weight of the skin care composition.

Thickeners may alter viscosity. Examples of useful thickeners in embodiments include plant-based polymer thickeners, mineral thickeners, wax thickeners, synthetic polymer thickeners, or combinations thereof. Plant-based polymer thickeners may include, without limitation, xanthan gum, konjac gum, guar gum, acacia gum, cellulose derivatives such as hydroxycellulose, hydroxyethyl cellulose, or combinations thereof. Mineral thickeners may include, without limitation, bentonite, magnesium aluminum silicate, or combinations thereof. Natural or synthetic waxes may include, without limitation, animal waxes, vegetable waxes, mineral waxes, petroleum waxes, silicone waxes, microcrystalline wax, or combinations thereof.

Synthetic polymer thickeners may include crosslinked polymers such as alkyl acrylate polymers in which the co-monomers consist of at least one of the following: acrylic acid, sodium acrylate, methacrylic acid, or alkyl acrylate. Polymers consisting purely of acrylic acid are often referred to as carbomers. Thus, for example, single molecules of carbomers can be crosslinked to provide a crosspolymer with a MW over 1 billion DA. In one example, acrylic acid may be crosslinked with allyl sucrose or allyl pentaerythritol. Similarly, a copolymer of C₁₀₋₃₀ alkyl acrylates and one or more monomers of acrylic acid, methacrylic acid or one of their simple esters may be crosslinked (e.g., with an allyl (2-propenyl) ether of sucrose or an allyl ether of pentaerythritol) to form acrylates/C₁₀₋₃₀ alkyl acrylate crosspolymer.

Thickeners may be present from about 0.01 wt. % to about 10.0 wt. %, based on the total weight of the personal care composition. A plant-based, mineral, wax, or synthetic polymer thickener may, for example, be present in an amount of 0.05 wt. %, 0.1 wt. %, 0.15 wt. %, 0.2 wt. %, 0.35 wt. %, 0.5 wt. %, 1.0 wt. %, 2.0 wt. %, 3.0 wt. %, 3.5 wt. %, 4.0 wt. %, 5.0 wt. %, 8.0 wt. %, 11.0 wt. %, including every number and/or fraction therebetween, based on the total weight of the personal care composition. In one example, a thickener is selected from hydroxyacetophenone, caprylic/capric triglyceride (and) polyurethane-79, trilaurin, PEG-150 pentaerythrityl tetrastearate (and) PEG-6 caprylic/capric glycerides (and) water, PEG-14M, microcrystalline wax, or combinations thereof.

Antioxidants may include, for example, carotenoids (e.g., beta-carotene, lycopene, lutein, zeaxanthin), additional polyphenols (e.g., resveratrol), selenium, vitamins, derivatives thereof, or combinations thereof. For example, a vitamin may be selected from vitamin A (retinoic acid), vitamin B (e.g., nicotinic acid or nicotinamide), vitamin C (ascorbic acid), vitamin E (tocopherol), or combinations thereof. Meanwhile, a vitamin derivative may be selected from esters of vitamin A (e.g., retinyl acetate, etc.), vitamin C (ascorbyl palmitate, etc.), vitamin E (e.g., tocopheryl acetate, tocopheryl palmitate, tocopheryl linoleate, etc.), or combinations thereof. Antioxidants may include, for example, grape seed extract, vitamin E, tocopherol, ubiquinone, vitamin C, vitamin A (retinol), vitamin B (e.g., B3 niacinamide), vitamin C (ascorbic acid), vitamin E (tocopherol), derivatives thereof, polyphenols (e.g., flavonoids), ferulic acid, resveratrol, green tea extract, extracts from fruit, berries, vegetables and nuts, Coenzyme Q10, or combinations thereof.

Antioxidants may be present from about 0.0001 wt. % to about 1.0 wt. %, based on the total weight of the personal care composition. For example, Camellia sinensis (green tea) extract may be present in an amount of 0.005 wt. %, 0.01 wt. %, 0.045, 0.098, 0.15 wt. %, 0.2 wt. %, 0.3 wt. %, 0.5 wt. %, 0.99 wt. %, including every number and/or fraction therebetween, based on the total weight of the personal care composition. Similarly, one or more additional antioxidants such as Daucus carota sativa (carrot) root extract or a vitamin or derivative thereof may be present in an amount of 0.0001 wt. %, 0.001 wt. %, 0.005 wt. %, 0.01 wt. %, 0.045, 0.098, 0.15 wt. %, 0.2 wt. %, 0.3 wt. %, 0.5 wt. %, including every number and/or fraction there between, based on the total weight of the personal care composition.

Example 2-in-1 Body Scrub and Shave Preparation Composition

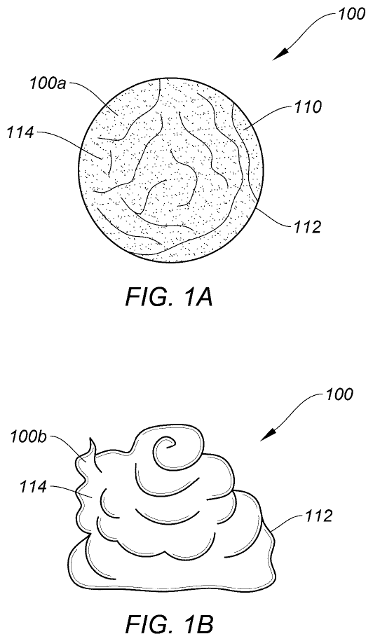

Referring now to FIGS. 1A-1B, an example of a topical composition 100 is formulated to transform from a body scrub 100a into a shave preparation 100b. In the example, the body scrub 100a includes a plurality of exfoliation particles 110 and a cosmetically acceptable carrier 112. The topical composition further includes a surfactant 114 mixed with the exfoliation particles 110 and the cosmetically acceptable carrier 112. The surfactant 114 is selected from an emulsifying surfactant, a cleansing surfactant, or combinations thereof.

The exfoliation particles 110 may be water-insoluble particles, water-soluble particles, or combinations thereof. For example, the water-insoluble particles may include a silica particle and the water-soluble particles may include a carbohydrate particle such as sucrose. The exfoliation particles 110 may have an average particle size from about 10 nm to about 1000 nm. In one example, the water-insoluble particle may be present in an amount of about 1.0 wt. % to about 10.0 wt. %, based on the total weight of the topical composition, the water-soluble particle may be present in an amount of about 10 wt. % to about 35.0 wt. %, based on the total weight of the topical composition, or combinations thereof.

In the illustrated example, the topical composition 100 may be in the form of an emulsion including an aqueous phase and a non-aqueous phase or may be in the form of an anhydrous base that is water-free or substantially water-free (e.g., <5.0 wt. % water). In one example, a total amount of water in an emulsion may present from about 30.0 wt. % to about 80.0 wt. %, preferably from about 50.0 wt. % to about 70.0 wt. %, based on the total weight of the topical composition. In addition, glycerin may be present in an amount of about 0.001 wt. % to about 1.0 wt. %, based on the total weight of the topical composition. However, in other aspects of embodiments, glycerin may be present in an amount up to about 15.0 wt. %, such as up to about 10.0 wt. %, including without limitation 1.5 wt. %, 2.0 wt. %, 5.0 wt. %, 7.0 wt. %, 10 wt. %, 11.0 wt. %, based on the total weight of the topical composition.

Meanwhile, a total amount of oil in an anhydrous base may present from about 30.0 wt. % to about 80.0 wt. %, preferably from about 30.0 wt. % to about 60.0 wt. %, based on the total weight of the topical composition. For example, Vitis vinifera (grape) seed oil may be present in an amount of about 10.0 wt. % to about 50.0 wt. %, based on the total weight of the topical composition, alone or together with one or more other oils. Additionally or alternatively, mineral oil and/or Cocos nucifera (coconut) oil may independently be present in an amount of about 1.0 wt. % to about 30.0 wt. %, preferably from about 5.0 wt. % to about 20.0 wt. %, based on the total weight of the topical composition.

Accordingly, in one example, a water-insoluble exfoliation particle may be dispersed in an emulsion. In another example, a water-insoluble exfoliation particle and/or a water-soluble exfoliation particle may be dispersed in an anhydrous base. However, other combinations, modifications, and variations fall within the true scope and spirit of the present disclosure. For example, a water-soluble exfoliation particle may be dispersed in an emulsion alone or together with a water-insoluble exfoliation particle. Ingredients and use levels to make a variety of formats may, therefore, be readily adjusted and mixed in a single phase or in multiple phases to provide a desired commercial personal care product.

Referring back to FIG. 1A, the surfactant 114 may include an emulsifying surfactant present in an emulsion in an amount of about 1.0 wt. % to about 8.0 wt. %, based on the total weight of the topical composition. The emulsifying surfactant may be the only type of surfactant present. Alternatively, the surfactant 114 may additionally include a cleansing surfactant present in an emulsion in an amount of about 1.0 wt. % to about 3.0 wt. %, based on the total weight of the topical composition. In either case, the topical composition 100 may provide relatively high exfoliation and/or relatively good but minimized shave comfort (e.g., where relatively less hair is shaved, where a relatively thinner product is preferred, etc.).

The surfactant 114 may include a cleansing surfactant. In an anhydrous base, for example, there may be enough oil in the body scrub 100a that the cleansing surfactant secondarily operates as an emulsifier when water mixes with the anhydrous base. Thus, no separate emulsifying surfactant is necessary. In contrast, a pre-emulsified body scrub 100a may require an emulsifying surfactant to stabilize the emulsion. In either case, however, a cleansing surfactant may be present to provide a lather effect to a pre-emulsified composition or to an emulsified composition created in situ or in real-time from an anhydrous base. A cleansing surfactant may be present in an amount of about 10.0 wt. % to about 60.0 wt. %, based on the total weight of the topical composition. For example, sodium lauryl sulfoacetate may be present in an amount about 10.0 wt. % to about 25.0 wt. %, sodium cocyl isethionate may be present in an amount of about 20.0 wt. % to about 40.0 wt. %, each based on the total weight of the topical composition, or combinations thereof. The shave preparation 100b may therefore provide relatively moderate to high exfoliation and/or relatively maximized shave comfort (e.g., where relatively more hair is shaved, a relatively thicker product is preferred, more shave preparation is preferred during a shave process, more glide is preferred, etc.).

In operation, a user applies the body scrub 100a on the skin of a body part. The user may rub the body scrub 100a on the skin during an exfoliation process. No water is required when, for example, the exfoliation particles 110 are water-insoluble particles (e.g., silica) and the topical composition 110 is in the form of an emulsion. As the user rubs the body scrub 100a on the skin, mechanical force (e.g., shear) breaks down the water-insoluble exfoliation particles. Over time, therefore, the water-insoluble exfoliation particles disappear and the texture of the body scrub 100a becomes less grainy. Without a sufficient amount of cleansing surfactant, the body scrub 100a also becomes thinner at a faster rate over time.

When the exfoliation process is complete, usually after at least a majority of water-insoluble particles are broken down or when the body scrub 100a does not feel substantially grainy but ultimately at the discretion of the consumer, the user may complete a shave operation by ending the rubbing step and applying a shave device (e.g., razor, etc.) on the skin to use the remaining topical composition 100 for at least some shave comfort. Thus, the topical composition 100 is formulated to have a sufficient amount of exfoliation particles that provide adequate exfoliation (e.g., not necessarily maximum exfoliation) which can be broken down within a sufficient amount of time so as to still allow the thinned topical composition 100 to remain and be used as a shave preparation after the exfoliation process.

In another example, a user applies the body scrub 100a on the skin of a body part. The user may rub the body scrub 100a, in the form of an anhydrous base, on the skin and contact the body scrub 100a with water during an exfoliation process before and/or after applying the body scrub 100a. As the user rubs the body scrub 100a on the skin, mechanical force (e.g., shear) breaks down any water-insoluble exfoliation particles and the water dissolves any water-soluble exfoliation particles. Moreover, the anhydrous base transforms into an emulsion owing to any emulsifying surfactant and/or cleansing surfactant present in the body scrub 100a. Over time, the exfoliation particles 110 disappear and the texture of the body scrub 100a becomes less grainy. With a sufficient amount of cleansing surfactant, the rate at which the body scrub 100a becomes thin is smaller (compared to a composition with substantially less cleansing surfactant) and/or the body scrub 100a lathers, foams, etc.

When the exfoliation process is complete, usually after at least a majority of the exfoliation particles 110 are broken down and dissolved or when the body scrub 100a does not feel substantially grainy but ultimately at the discretion of the consumer, the user may complete a shave operation by ending the rubbing step and applying a shave device (e.g., razor, etc.) on the skin to use the shave preparation 100b for maximized shave comfort (e.g., moderate to high glide). Thus, the shave preparation 100b remains on the skin for a relatively longer period of time and/or provides better shave comfort during the shave process compared to a topical composition having exfoliation particles but with less surfactant or no cleansing surfactant that is used for the same period of time. It is understood, however, that other combinations, modifications, and variations fall within the true scope and spirit of the present disclosure. For example, an emulsion may include a cleansing surfactant in a sufficient amount to provide a later effect when contacted with water.

The following examples demonstrate non-limiting aspects of the topical composition 100. The following examples were prepared using traditional formulation processes, generally consisting of mixing the ingredients shown in the following examples at the shown use levels in a single phase or in multiple phases to manufacture the following examples.

### Example Emulsion Composition

Table 1 shows topical compositions having exfoliation particles, a cosmetically acceptable carrier, an emulsifying surfactant, a cleansing surfactant, and ingredients such as thickeners, antioxidants, opacifiers, preservatives, etc. The topical compositions in Table 1 are formulated to have a sufficient amount of exfoliation particles that provide adequate exfoliation (e.g., not necessarily maximum exfoliation) which can be broken down within a sufficient amount of time so as to still allow a resulting thinned composition to remain on the skin and be used as shave preparations for shave comfort during a subsequent shave process.

TABLE 1
Topical Compositions with Lower Levels of Cleansing Surfactant.

| Component (INCI) | wt. % (±10%) |
|---|---|
| Example Exfoliation Particle | |
| Silica | 1.0-10.0 |
| Example Carrier | |
| Aqua (Water) | 50.0-70.0 |
| Glycerin | 5.0-10.0 |
| Hydrogenated Vegetable Oil (and) | 0 .5-13.0 |
| Olea Europaea (Olive) Fruit Oil, | |
| Dimethicone, Isopropyl Palmitate, | |
| Mineral Oil, Petrolatum, | |
| Butyrospermum Parkii (Shea) Butter, | |
| Glycereth-18 Ethylhexanoate | |
| (and) Glycereth-18 | |
| Example Surfactant | |
| Behentrimonium Methosulfate (and) | 1.0-3.0 |
| Cetearyl Alcohol | |
| Stearyl Alcohol, Cetyl Alcohol, Cetearyl Alcohol | 0.5-4.0 |
| Example Other Ingredients | |
| Hydroxyacetophenone, PEG-150 | 0.001-4.0 |
| Pentaerythrityl Tetrastearate (and) | |
| PEG-6 Caprylic/Capric Glycerides | |
| (and) Water, PEG-14M, Titanium | |
| Dioxide, Tocopheryl Acetate, | |
| Hydrolyzed Soy Protein, Camellia | |
| Sinensis Leaf Extract, Aloe Barbadensis | |
| Leaf Juice, Phenoxyethanol, | |
| Fragrance | |

Table 2 shows topical compositions similar to the topical compositions shown in Table 1, except notably that exfoliating particles are present in relatively lower amounts and the cleansing surfactants are present in relatively higher amounts. The shave preparations formed during an exfoliation process using the topical compositions in Table 2 remain on the skin for a relatively longer period of time and/or provide better shave comfort during a shave process subsequent to the exfoliation process compared to the compositions in Table 1.

TABLE 2
Topical Compositions with Higher Levels of Cleansing Surfactant.

| Component (INCI) | wt. % (±10%) |
|---|---|
| Example Exfoliation Particle | |
| Silica | 1.0-5.0 |
| Example Carrier | |
| Aqua (Water) | 50.0-60.0 |
| Glycerin | 1.0-3.5 |
| Hydrogenated Vegetable Oil (and) | 0.2-4.5 |
| Olea Europaea (Olive) Fruit Oil, | |
| Dimethicone, Isopropyl Palmitate, | |
| Mineral Oil, Petrolatum, | |
| Butyrospermum Parkii (Shea) Butter, | |
| Glycereth-18 Ethylhexanoate | |
| (and) Glycereth-18 | |
| Example Surfactant | |
| Sodium Cocoyl Isethionate | 30.0-40.0 |
| Behentrimonium Methosulfate (and) | 0.5-1.0 |
| Cetearyl Alcohol | |
| Stearyl Alcohol, Cetyl Alcohol, Cetearyl Alcohol | 0.5-4.0 |
| Example Other Ingredients | |
| Hydroxyacetophenone, PEG-150 | 0.001-4.0 |
| Pentaerythrityl Tetrastearate (and) | |
| PEG-6 Caprylic/Capric Glycerides | |
| (and) Water, PEG-14M, Titanium | |
| Dioxide, Tocopheryl Acetate, | |
| Hydrolyzed Soy Protein, Camellia | |
| Sinensis Leaf Extract, Aloe Barbadensis | |
| Leaf Juice, Phenoxyethanol, | |
| Fragrance. | |

Table 3 shows topical compositions having exfoliation particles, a cosmetically acceptable carrier, an emulsifying surfactant, and ingredients such as thickeners, antioxidants, preservatives, etc. Notably, the exfoliation particles include both water-insoluble particles and water-soluble particles. The topical compositions in Table 3 are formulated to have a sufficient amount of exfoliation particles that provide adequate exfoliation (e.g., not necessarily maximum exfoliation) which can be broken down or dissolved within a sufficient amount of time so as to still allow the resulting thinned topical compositions to remain on the skin and be used as shave preparations for shave comfort.

TABLE 3
Topical Compositions with Water-Insoluble Particles, Water-Insoluble
Particles, and Emulsifying Surfactant Only.

| Component (INCI) | wt. % (±10%) |
|---|---|
| Example Exfoliation Particle | |
| Silica | 1.0-5.0 |
| Sucrose | 5.0-10.0 |
| Example Carrier | |
| Aqua (Water) | 60.0-70.0 |
| Cocos Nucifera (Coconut) Oil | 5.0-10.0 |
| Vitis Vinifera (Grape) Seed Oil | 1.0-5.0 |
| Hydrogenated Vegetable Oil (and) | 0.01-2.25 |
| Olea Europaea (Olive) Fruit Oil, | |
| Butyrospermum Parkii (Shea) | |
| Butter, Mangifera Indica (Mango) | |
| Seed Butter | |
| Example Surfactant | |
| Stearyl Alcohol, Cetyl Alcohol, | 0.5-4.0 |
| Cetearyl Alcohol, Ceteareth-20 | |
| Example Other Ingredients | |
| Panthenol (and) Water, Tocopheryl | 0.0001-1.5 |
| Acetate, Aloe Barbadensis Leaf | |
| Juice, Raphanus Sativus (Radish) | |
| Root Extract (and) Citric Acid (and) | |
| Maltodextrin, Phenoxyethanol (and) Ethylhexylglycerin | |

Table 4 shows topical compositions having exfoliation particles, a cosmetically acceptable carrier, a cleansing surfactant, and ingredients such as thickeners, antioxidants, preservatives, etc. Notably, the shave preparations formed during an exfoliation process using the topical compositions in Table 4 remain on the skin for a relatively longer period of time and/or provide better shave comfort (e.g., moderate to high glide, etc.) during a shave process subsequent to the exfoliation process compared to the compositions in Tables 1-2.

TABLE 4
Topical Composition with Higher Levels of Cleansing Surfactant.

| Component (INCI) | wt. % (±10%) |
|---|---|
| Example Exfoliation Particle | |
| Silica | 1.0-5.0 |
| Example Carrier | |
| Aqua (Water) | 45.0-55.0 |
| Sorbitol | 5.0-10.0 |
| Benzyl Alcohol, Hydrogenated | 0.5-5.5 |
| Vegetable Oil (and) Olea Europaea | |
| (Olive) Fruit Oil, Butyrospermum Parkii | |
| (Shea) Butter, Glycereth-18 | |
| Ethylhexanoate (and) Glycereth-18 | |
| Example Surfactant | |
| Sodium Cocoyl Isethionate | 20.0-30.0 |
| Example Other Ingredients | |
| Hydroxyacetophenone, Tocopheryl | 0.1-0.1 |
| Acetate, Hydrolyzed Soy Protein, | |
| Camellia Sinensis Leaf Extract, | |
| Lactobacillus (and) Cocos Nucifera | |
| (Coconut) Fruit Extract, Aloe Barbadensis Leaf Juice, | |
| Phenoxyethanol, Fragrance | |

### Example Anhydrous Composition

Table 5 shows topical compositions having exfoliation particles, a cosmetically acceptable carrier, a cleansing surfactant, and ingredients such as thickeners and fragrance. The shave preparations formed during an exfoliation process using the topical compositions in Table 5 remain on the skin for a relatively long period of time and/or provide relatively good shave comfort (e.g., moderate to high glide, etc.) during a shave process subsequent to the exfoliation process.

TABLE 5
Topical Composition with Higher Levels of Cleansing Surfactant.

| Component (INCI) | wt. % (±10%) |
|---|---|
| Example Exfoliation Particle | |
| Sucrose | 15.0-25.0 |
| Example Carrier | |
| Vitis Vinifera (Grape) Seed Oil | 30.0-40.0 |
| Cocos Nucifera (Coconut) Oil | 0.0-7.5 |
| Glycerin | 0.0-5.0 |
| Hydrogenated Vegetable Oil (and) | 0.0-1.0 |
| Olea Europaea (Olive) Fruit Oil, | |
| Butyrospermum Parkii (Shea) | |
| Butter, Mangifera Indica (Mango) | |
| Seed Butter | |
| Example Surfactant | |
| Sodium Cocoyl Isethionate | 20.0-30.0 |
| Sodium Lauryl Sulfoacetate | 10.0-20.0 |
| Polysorbate-20 | 0.0-3.0 |
| Example Other Ingredients | |
| Caprylic/Capric Triglyceride (and) | 0.2-3.0 |
| Polyurethane-79, Trilaurin, | |
| Fragrance | |

Table 6 shows topical compositions having exfoliation particles, a cosmetically acceptable carrier, a cleansing surfactant, and ingredients such as fragrance. The shave preparations formed during an exfoliation process using the topical compositions in Table 6 may remain on the skin for a relatively long period of time and/or provide relatively good shave comfort during a shave process subsequent to the exfoliation process.

TABLE 6
Topical Composition with Higher Levels of Cleansing Surfactant.

| Component (INCI) | wt. % (±10%) |
|---|---|
| Example Exfoliation Particle | |
| Sucrose | 30.0-40.0 |
| Example Carrier | |
| Vitis Vinifera (Grape) Seed Oil | 15.0-20.0 |
| Cocos Nucifera (Coconut) Oil | 15.0-20.0 |
| Glycerin | 0.1-1.0 |
| Mangifera Indica (Mango) Seed Butter | 0.1-3.0 |
| Example Surfactant | |
| Sodium Cocoyl Isethionate | 20.0-30.0 |
| Polysorbate-20 | 1.0-3.0 |
| Example Other Ingredients | |
| Fragrance | 0.1-1.0 |

Table 7 shows topical compositions having exfoliation particles, a cosmetically acceptable carrier, a cleansing surfactant, and ingredients such as thickeners and fragrance. Topical compositions (not shown) were also prepared with sodium cocoyl isethionate in an amount of about 10.0 wt. % to about 15.0 wt. %, based on the total weight of the topical composition. The shave preparations formed during an exfoliation process using these compositions may remain on the skin for a relatively long period of time and/or provide relatively good shave comfort during a shave process subsequent to the exfoliation process.

TABLE 7
Topical Composition with Lower to Higher Levels of Cleansing Surfactant.

| Component (INCI) | wt. % (±10%) |
|---|---|
| Example Exfoliation Particle | |
| Sucrose | 10.0-25.0 |
| Example Carrier | |
| Vitis Vinifera (Grape) Seed Oil | 50.0-85.0 |
| Example Surfactant | |
| Sodium Lauryl Sulfoacetate | 2.0-20.0 |
| Example Other Ingredients | |
| Caprylic/Capric Triglyceride (and) | 0.5-6.0 |
| Polyurethane-79, Trilaurin, Fragrance | |

Table 8 shows topical compositions having exfoliation particles, a cosmetically acceptable carrier, and a cleansing surfactant. The shave preparations formed during an exfoliation process using the topical compositions in Table 8 may remain on the skin for a relatively long period of time and/or provide relatively good shave comfort during a shave process subsequent to the exfoliation process.

TABLE 8
Topical Composition with Higher Levels of Cleansing Surfactant.

| Component (INCI) | wt. % (±10%) |
|---|---|
| Example Exfoliation Particle | |
| Sucrose | 15.0-25.0 |
| Example Carrier | |
| Vitis Vinifera (Grape) Seed Oil | 10.0-20.0 |
| Cocos Nucifera (Coconut) Oil | 40.0-60.0 |
| Example Surfactant | |
| Sodium Lauryl Sulfoacetate | 10.0-20.0 |

Table 9 shows topical compositions having exfoliation particles, a cosmetically acceptable carrier, a cleansing surfactant, and other ingredients such as thickeners. The shave preparations formed during an exfoliation process using the topical compositions in Table 9 may remain on the skin for a relatively long period of time and/or provide relatively good shave comfort during a shave process subsequent to the exfoliation process.

TABLE 9
Topical Composition with Higher Levels of Cleansing Surfactant.

| Component (INCI) | wt. % (±10%) |
|---|---|
| Example Exfoliation Particle | |
| Sucrose | 20.0-30.0 |
| Example Carrier | |
| Vitis Vinifera (Grape) Seed Oil | 50.0-60.0 |
| Example Surfactant | |
| Sodium Lauryl Sulfoacetate | 5.0-10.0 |
| Decyl Glucoside | 10.0-20.0 |
| Example Other Ingredients | |
| Caprylic/Capric Triglyceride (and) Polyurethane-79 | 2.5-7.5 |

Table 10 shows topical compositions having exfoliation particles, a cosmetically acceptable carrier, a cleansing surfactant, and ingredients such as thickeners, antioxidants, preservatives, etc. The shave preparations formed during an exfoliation process using the topical compositions in Table 10 may remain on the skin for a relatively long period of time and/or provide relatively good shave comfort during a shave process subsequent to the exfoliation process.

TABLE 10
Topical Composition with Water-Insoluble Particles, Water-Insoluble
Particles, and Higher Levels of Cleansing Surfactant.

| Component (INCI) | wt. % (±10%) |
|---|---|
| Example Exfoliation Particle | |
| Silica | 1.0-5.0 |
| Sucrose | 30.0-40.0 |
| Example Carrier | |
| Cocos Nucifera (Coconut) Oil | 15.0-20.0 |
| Vitis Vinifera (Grape) Seed Oil | 15.0-20.0 |
| Glycerin | 0.1-1.0 |
| Hydrogenated Vegetable Oil (and) | 0.02-3.0 |
| Olea Europaea (Olive) Fruit Oil, | |
| Butyrospermum Parkii (Shea) | |
| Butter, Mangifera Indica (Mango) | |
| Seed Butter | |
| Example Surfactant | |
| Sodium Lauryl Isethionate | 10.0-20.0 |
| Polysorbate-20 | 1.0-7.5 |
| Example Other Ingredients | |
| Panthenol (and) Water, Tocopheryl | 0.01-1.5 |
| Acetate, Aloe Barbadensis Leaf | |
| Juice, Carica Papaya (Papaya) | |
| Extract, Phenoxyethanol (and) | |
| Ethylhexylglycerin, Fragrance | |

Table 11 shows topical compositions having exfoliation particles, a cosmetically acceptable carrier, a cleansing surfactant, and ingredients such as thickeners, fragrance, preservatives, and antioxidants. The sucrose includes a mixture of white and brown sucrose. Moreover, mineral oil is used to offset for a reduction of other oils such as Vitis Vinifera (Grape) Seed Oil and microcrystalline wax is used as a thickener. The shave preparations formed during an exfoliation process using the topical compositions in Table 11 remain on the skin for a relatively long period of time and/or provide relatively good shave comfort (e.g., moderate to high glide, etc.) during a shave process subsequent to the exfoliation process.

TABLE 11
Topical Composition with Higher Levels of Cleansing Surfactant.

| Component (INCI) | wt. % (±10%) |
|---|---|
| Example Exfoliation Particle | |
| Sucrose | 20.0-30.0 |
| Example Carrier | |
| Vitis Vinifera (Grape) Seed Oil | 10.0-20.0 |
| Cocos Nucifera (Coconut) Oil | 5.0-12.0 |
| Mineral Oil | 5.0-12.0 |
| Glycerin | 1.0-11.0 |
| Butyrospermum Parkii (Shea) | 0.1-11.0 |
| Butter, Mangifera Indica (Mango) | |
| Seed Butter | |
| Example Surfactant | |
| Sodium Cocoyl Isethionate | 20.0-30.0 |
| Sodium Lauryl Sulfoacetate | 8.0-20.0 |
| Polysorbate-20 | 1.0-3.0 |
| Example Other Ingredients | |
| Caprylic/Capric Triglyceride, | 0.1-3.0 |
| Microcrystalline Wax, Trilaurin | |
| Phenoxyethanol (and) Ethylhexylglycerin, | 0.2-1.5 |
| Tocopheryl Acetate, | |
| Fragrance | |

### Example Sensory Analysis

Descriptive sensory analysis was conducted using panelists trained to detect and quantify sensations related to scrub and shave performance of embodiments of topical compositions. Representative topical compositions listed in Table 5 were tested on a seven-point quantitative scale and scores were averaged for each attribute.

A first topical composition tested by fifty-four panelists consisted of sucrose, Vitis vinifera (Grape) seed oil, Cocos nucifera (Coconut) oil, Mangifera indica (Mango) seed butter, caprylic/capric triglyceride (and) polyurethane-79, polysorbate-20, glycerin, fragrance, sucrose, trilaurin, sodium cocoyl isethionate, and sodium lauryl sulfoacetate. The topical composition scored high for overall attributes as follows: overall liking (87%); overall liking for scrub (92%); overall liking shave preparation (80%); overall performance (87%). After two shaves, the topical composition also scored high for performance attributes as follows: ease of dispense (87%); ease of spread for scrub (86%); exfoliated skin (94%); ease to spread shave preparation (82%); ease of lather (82%); tracking (83%); reduce skin irritation (88%); no nicks and cuts (88%); comfortable shave (87%); close shave (87%); prevents pulling and tugging (87%); prevents razor bumps (88%); improves razor glide (80%). The skin related attributes also scored high as follows: reduced redness (87%); soft skin (93%); smooth skin (93%); moisturized skin (91%); rinse off skin (86%).

A second representative topical composition tested by fifty-seven panelists consisted of sucrose, Vitis vinifera (Grape) seed oil, caprylic/capric triglyceride (and) polyurethane-79, fragrance, sucrose, trilaurin, sodium cocoyl isethionate, and sodium lauryl sulfoacetate. The topical composition scored high for overall attributes as follows: overall liking (88%); overall liking for scrub (92%); overall liking shave preparation (85%); overall performance (87%). After two shaves, the topical composition also scored high for performance attributes as follows: ease of dispense (82%); ease of spread for scrub (84%); exfoliated skin (91%); ease to spread shave preparation (82%); ease of lather (74%); tracking (76%); reduce skin irritation (85%); no nicks and cuts (85%); comfortable shave (86%); close shave (84%); prevents pulling and tugging (86%); prevents razor bumps (86%); improves razor glide (81%). The skin related attributes also scored high as follows: reduced redness (85%); soft skin (90%); smooth skin (90%); moisturized skin (86%); rinse off skin (90%).

A third representative topical composition tested by fifty panelists consisted of sucrose, Vitis vinifera (Grape) seed oil, Cocos nucifera (Coconut) oil, Mangifera indica (Mango) seed butter, hydrogenated vegetable oil (and) Olea europaea (Olive) fruit oil, Butyrospermum parkii (Shea) butter, caprylic/capric triglyceride (and) polyurethane-79, fragrance, sucrose, trilaurin, sodium cocoyl isethionate, and sodium lauryl sulfoacetate. The topical composition scored moderately for overall attributes as follows: overall liking (72%); overall liking for scrub (72%); overall liking shave preparation (75%); overall performance (72%). After two shaves, the topical composition scores were mixed for performance attributes as follows: ease of dispense (44%); ease of spread for scrub (49%); exfoliated skin (76%); ease to spread shave preparation (67%); ease of lather (67%); tracking (81%); reduce skin irritation (85%); no nicks and cuts (85%); comfortable shave (86%); close shave (85%); prevents pulling and tugging (86%); prevents razor bumps (84%); improves razor glide (84%). The skin related attributes scored high as follows: reduced redness (84%); soft skin (89%); smooth skin (89%); moisturized skin (87%); rinse off skin (90%).

Given the results, there may be relatively good and tailored exfoliation, particle dissolution, lather, residence time on skin, and/or shave comfort. Moreover, there was no solidification or product separation with particle settlement of the first topical composition when tested for stability under various light and temperature conditions over several months. Accordingly, embodiments of topical compositions may be formulated to be stable and to transform from a body scrub into a shave preparation to minimize the unnecessary inconvenience to consumers or waste of resources from using multiple products.

### Additional Notes and Examples

Additional Example 1 includes a topical composition comprising an exfoliation particle including a water-insoluble particle, a water-soluble particle, or combinations thereof, and comprising a cosmetically acceptable carrier, wherein the topical composition is in the form of an emulsion including an aqueous phase and a non-aqueous phase, wherein the water-insoluble particle is dispersed in the emulsion, or wherein the topical composition is in the form of an anhydrous base, wherein one or more of the water-insoluble particle or the water-soluble particle is dispersed in the anhydrous base, and wherein the emulsion and the anhydrous base optionally further include a cleansing surfactant.

Additional Example 2 includes the topical composition of Additional Example 1, wherein the emulsion and the anhydrous base include the cleansing surfactant present in an amount of about 10.0 wt. % to about 50.0 wt. %, based on the total weight of the topical composition.

Additional Example 3 includes the topical composition of any of Additional Examples 1-2, wherein the water-insoluble particle comprises a silica particle and the water-soluble particle comprises a carbohydrate particle.

Additional Example 4 includes the topical composition of any of Additional Examples 1-3, wherein the silica particle has an average particle size from about 10 nm to about 500 m.

Additional Example 5 includes the topical composition of any of Additional Examples 1-4, wherein the carbohydrate particle comprises a sucrose particle.

Additional Example 6 includes the topical composition of any of Additional Examples 1-5, wherein the sucrose particle has an average particle size from about 450 nm to about 1000 nm.

Additional Example 7 includes the topical composition of any of Additional Examples 1-6, wherein the water-insoluble particle is present in an amount of about 1.0 wt. % to about 10.0 wt. %, based on the total weight of the topical composition, the water-soluble particle is present in an amount of about 2.0 wt. % to about 35.0 wt. %, based on the total weight of the topical composition, or combinations thereof.

Additional Example 8 includes the topical composition of any of Additional Examples 1-7, wherein the emulsion further includes an emulsifying surfactant and glycerin in an amount of about 0.01 wt. % to about 1.0 wt. %, based on the total weight of the topical composition.

Additional Example 9 includes the topical composition of any of Additional Examples 1-8, wherein the anhydrous base comprises an oil.

Additional Example 10 includes the topical composition of any of Additional Examples 1-9, wherein the oil comprises Vitis vinifera (grape) seed oil, Cocos nucifera (coconut) oil, mineral oil, or combinations thereof.

Additional Example 11 includes the topical composition of any of Additional Examples 1-10, wherein the Vitis vinifera (grape) seed oil is present in an amount of about 10.0 wt. % to about 50.0 wt. %, based on the total weight of the topical composition.

Additional Example 12 includes the topical composition of any of Additional Examples 1-11, wherein the Cocos nucifera (coconut) oil is present in an amount of about 5.0 wt. % to about 20.0 wt. %, based on the total weight of the topical composition.

Additional Example 13 includes the topical composition of any of Additional Examples 1-12, wherein the mineral oil is present in an amount of about 5.0 wt. % to about 20.0 wt. %, based on the total weight of the topical composition.

Additional Example 14 includes the topical composition of any of Additional Examples 1-13, wherein the cleansing surfactant comprises sodium cocyl isethionate, sodium lauryl sulfoacetate, polysorbate-20, decyl glucoside, behentrimonium methosulfate, or combinations thereof.

Additional Example 15 includes the topical composition of any of Additional Examples 1-14, wherein the cleansing surfactant comprises sodium lauryl sulfoacetate present in an amount about 10.0 wt. % to about 20.0 wt. %, based on the total weight of the topical composition.

Additional Example 16 includes the topical composition of any of Additional Examples 1-15, wherein the cleansing surfactant comprises sodium cocyl isethionate present in an amount of about 20.0 wt. % to about 40.0 wt. %, based on the total weight of the topical composition.

Additional Example 17 includes the topical composition of any of Additional Examples 1-16, wherein the cleansing surfactant comprises polysorbate-20 present in an amount of about 1.0 wt. % to about 3.0 wt. %, based on the total weight of the topical composition.

Additional Example 18 includes the topical composition of any of Additional Examples 1-17, wherein the exfoliation particle comprises a water-soluble particle, wherein the topical composition is in the form of an anhydrous base, wherein the water-soluble particle is dispersed in the anhydrous base, and wherein the anhydrous base further includes a cleansing surfactant present in an amount of about 10.0 wt. % to about 50.0 wt. %, based on the total weight of the topical composition.

Additional Example 19 includes the topical composition of any of Additional Examples 1-18, wherein the exfoliation particle comprises a water-insoluble particle present in an amount of about 1.0 wt. % to about 10.0 wt. %, based on the total weight of the topical composition, wherein the topical composition is in the form of an emulsion including an aqueous phase, a non-aqueous phase, and a surfactant, and wherein the water-insoluble particle is dispersed in the emulsion.

Additional Example 20 includes the topical composition of any of Additional Examples 1-19, wherein the surfactant includes an emulsifying surfactant and excludes a cleaning surfactant.

Additional Example 21 includes the topical composition of any of Additional Examples 1-20, wherein the surfactant includes a cleansing surfactant present in an amount of about 1.0 wt. % to about 3.0 wt. %, based on the total weight of the topical composition.

Additional Example 22 includes the topical composition of any of Additional Examples 1-21, further including one or more other ingredients selected from water, film formers, thickeners, antioxidants, amino acids, colorants, opacifiers, pH adjusters, fragrances, preservatives, preservative boosters, chelating agents, stabilizers, minerals, anti-inflammatory agents, anti-irritants, anti-acne agents, skin cooling compounds, skin penetration enhancers, extracts, vitamins, or combinations thereof.

Additional Example 23 includes the topical composition of any of Additional Examples 1-22, wherein the thickener is selected from the group consisting of caprylic/capric triglyceride, microcrystalline wax, trilaurin, polyurethane-79, or combinations thereof.

Additional Example 24 includes the topical composition of any of Additional Examples 1-23, wherein the water-insoluble particle is present in an amount of about 1.0 wt. % to about 10.0 wt. % and/or the water-soluble particle is present in an amount of about 20.0 wt. % to about 30.0 wt. %, wherein the topical composition is in the form of an anhydrous base, wherein the anhydrous base comprises an oil present in an amount of about 30.0 wt. % to about 60.0 wt. %, wherein Vitis vinifera (grape) seed oil is present in an amount of about 10.0 wt. % to about 20.0 wt. %, wherein Cocos nucifera (coconut) oil is present in an amount of about 5.0 wt. % to about 20.0 wt. %, wherein mineral oil is present in an amount of about 5.0 wt. % to about 20.0 wt. %, wherein sodium lauryl sulfoacetate is present in an amount of about 10.0 wt. % to about 20.0 wt. %, wherein sodium cocyl isethionate is present in an amount of about 20.0 wt. % to about 40.0 wt. %, wherein polysorbate-20 is present in an amount of about 1.0 wt. % to about 3.0 wt. %, and wherein the thickener is selected from the group consisting of caprylic/capric triglyceride, microcrystalline wax, trilaurin, polyurethane-79, or combinations thereof.

Additional Example 25 includes a packaged consumer product comprising the topical composition of any of Additional Examples 1-24.

Additional Example 26 includes a method to exfoliate skin of a body part during an exfoliation process and to provide shave comfort to the skin of the body part during a shave process comprising contacting the skin with the topical composition of any of Additional Examples 1-24, rubbing the topical composition on the skin to exfoliate the skin during the exfoliation process, and applying a shave device to the skin during the shave process subsequent to the exfoliation process.

Additional Example 27 includes the method of Additional Example 26, further including contacting the topical composition with water, rubbing the topical composition on the skin for a period of time within which one or more of mechanical force breaks down a majority of water-insoluble particles or the water dissolves a majority of the water-soluble particles and the cleansing surfactant mixes with the water to provide a lather effect that causes the topical composition to remain on the skin and provide better shave comfort during the shave process compared to a topical composition having the exfoliation particles with less surfactant or no cleansing surfactant that is used for the same time.

Additional Example 28 includes a method to make a topical composition to exfoliate skin of a body part during an exfoliation process and to subsequently provide shave comfort to the skin of the body part during a shave process comprising mixing ingredients of any of Additional Examples 1-24.

In this disclosure and the following claims, the word “or” indicates an inclusive list such that, for example, the list of X, Y, or Z means X or Y or Z or XY or XZ or YZ or XYZ. Similarly, the list of one or more of X, Y, or Z or a list of X, Y, Z, or combinations thereof means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

While the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

1. A topical composition comprising:
an exfoliation particle comprising a water-insoluble particle, a water-soluble particle, or combinations thereof; and
a cosmetically acceptable carrier;
wherein the topical composition is in the form of an anhydrous base;
wherein one or more of the water-insoluble particle or the water-soluble particle is dispersed in the anhydrous base; and
wherein the anhydrous base further includes a cleansing surfactant present in an amount of about 10.0 wt. % to about 50.0 wt. %, based on the total weight of the topical composition.

2. The topical composition of claim 1, wherein the water-insoluble particle comprises a silica particle and the water-soluble particle comprises a carbohydrate particle.

3. The topical composition of claim 2, wherein the silica particle has an average particle size from about 10 nm to about 500 nm.

4. The topical composition of claim 2, wherein the carbohydrate particle comprises a sucrose particle.

5. The topical composition of claim 4, wherein the sucrose particle has an average particle size from about 450 nm to about 1000 nm.

6. The topical composition of claim 1, wherein the water-insoluble particle is present in an amount of about 1.0 wt. % to about 10.0 wt. %, based on the total weight of the topical composition, the water-soluble particle is present in an amount of about 20.0 wt. % to about 30.0 wt. %, based on the total weight of the topical composition, or combinations thereof.

7. The topical composition of claim 1, wherein the anhydrous base comprises an oil.

8. The topical composition of claim 7, wherein a total amount of oil present is from about 30.0 wt. % to about 60.0 wt. %, based on the total weight of the topical composition.

9. The topical composition of claim 7, wherein the oil comprises Vitis vinifera (grape) seed oil, Cocos nucifera (coconut) oil, mineral oil, or combinations thereof.

10. The topical composition of claim 9, wherein the Vitis vinifera (grape) seed oil is present in an amount of about 10.0 wt. % to about 20.0 wt. %, based on the total weight of the topical composition.

11. The topical composition of claim 9, wherein the Cocos nucifera (coconut) oil is present in an amount of about 5.0 wt. % to about 20.0 wt. %, based on the total weight of the topical composition.

12. The topical composition of claim 9, wherein the mineral oil is present in an amount of about 5.0 wt. % to about 20.0 wt. %, based on the total weight of the topical composition.

13. The topical composition of claim 1, wherein the cleansing surfactant comprises sodium cocyl isethionate, sodium lauryl sulfoacetate, polysorbate-20, decyl glucoside, behentrimonium methosulfate, or combinations thereof.

14. The topical composition of claim 13, wherein the cleansing surfactant comprises sodium lauryl sulfoacetate present in an amount about 10.0 wt. % to about 20.0 wt. %, based on the total weight of the topical composition.

15. The topical composition of claim 13, wherein the cleansing surfactant comprises sodium cocyl isethionate present in an amount of about 20.0 wt. % to about 40.0 wt. %, based on the total weight of the topical composition.

16. The topical composition of claim 13, wherein the cleansing surfactant comprises polysorbate-20 present in an amount of about 1.0 wt. % to about 3.0 wt. %, based on the total weight of the topical composition.

17. The topical composition of claim 1, further including one or more other ingredients selected from water, film formers, thickeners, antioxidants, amino acids, colorants, opacifiers, pH adjusters, fragrances, preservatives, preservative boosters, chelating agents, stabilizers, minerals, anti-inflammatory agents, anti-irritants, anti-acne agents, skin cooling compounds, skin penetration enhancers, extracts, vitamins, or combinations thereof.

18. The topical composition of claim 17, wherein the thickener is selected from the group consisting of caprylic/capric triglyceride, microcrystalline wax, trilaurin, polyurethane-79, or combinations thereof.

19. A packaged consumer product comprising the topical composition of claim 1.

20. (canceled)

21. A method of making topical composition comprising:
mixing an exfoliation particle comprising a water-insoluble particle, a water-soluble particle, or combinations thereof, and a cosmetically acceptable carrier;
wherein the topical composition is in the form of an anhydrous base;
wherein one or more of the water-insoluble particle or the water-soluble particle is dispersed in the anhydrous base; and
wherein the anhydrous base further includes a cleansing surfactant present in an amount of about 10.0 wt. % to about 50.0 wt. %, based on the total weight of the topical composition.
